(19) United States
(12) Patent Application Publication
(10) Pub. No.: US 2023/0149513 A1
(43) Pub. Date: May 18, 2023

(54) PHARMACEUTICAL COMPOSITION FOR CORNEAL TISSUE REPAIR BY INSULIN NANOSYSTEM AND USE THEREOF

(71) Applicant: The Second Affiliated Hospital of Chongqing Medical University, Chongqing (CN)

(72) Inventor: Zhiyu DU, Chongqing (CN)

(21) Appl. No.: 18/054,951

(22) Filed: Nov. 14, 2022

(30) Foreign Application Priority Data
Nov. 15, 2021 (CN) 202111347384.0

(51) Int. Cl. A61K 38/28; A61K 9/127; A61P 27/02

(52) U.S. Cl. CPC A61K 38/28; A61K 9/127; A61P 27/02

(57) ABSTRACT

The present disclosure provides a pharmaceutical composition for corneal tissue repair by an insulin nanosystem and use thereof, and belongs to the technical field of pharmaceutical compositions. Key points of technical solutions of the pharmaceutical composition are as follows: including such nanomaterials as liposome and insulin encapsulated in the nanomaterials like liposome; and using the pharmaceutical composition for corneal tissue repair by an insulin nanosystem in corneal repair. The present disclosure is mainly intended to provide a novel ophthalmic pharmaceutical composition, which has the advantages of safety, effectiveness, low side effects and low administration frequency, and can effectively improve patient compliance; meanwhile, the ophthalmic pharmaceutical composition has important biological functions in anti-inflammation, anti-apoptosis and promoting corneal cell and nerve repairs, and has more advantages in the treatment effects of anti-inflammation, anti-infection, promoting corneal repair and strengthening amniotic membrane transplantation compared with a traditional treatment method.

### TECHNICAL FIELD

The present disclosure relates to the technical field of pharmaceutical compositions, in particular to a pharmaceutical composition for corneal tissue repair by an insulin nanosystem and use thereof.

### BACKGROUND

Corneal injuries of different etiologies, such as severe dry eye, infectious or non-infectious keratitis, chemical burns, corneal transplantation rejection, poor repair after various keratoplastic surgeries, and corneal trauma, remain the main and common clinical corneal diseases and disorders. Despite corresponding treatment, some patients are still unsatisfied with the efficacy, resulting in refractory corneal diseases. In this case, the infiltration of some cytokines and inflammatory factors in the tear and corneal tissue may lead to delayed corneal epithelial healing, recurrent corneal ulcers, neovascularization, edema, corneal opacity, and decreased visual acuity. Therefore, it is particularly important for the treatment of similar corneal diseases.

At present, main therapies used include anti-inflammation, anti-infection, administration of growth factors that promote corneal repair, and amniotic membrane transplantation. Topical administration of corticosteroids is beneficial to inhibit the development of inflammation, and amniotic membrane transplantation and various growth factors are beneficial to promote the repair and reconstruction of corneal tissue. Topical dropping is the preferred non-invasive anterior ocular segment administration route, and 90% of conventional ophthalmic preparations on the market are eye drops. However, the bioavailability of the topical dropping is relatively low, because the residence time and penetration of the drug are affected by a plurality of anatomical and physiological factors, for example: blinking, tear secretion, and nasolacrimal duct drainage make the drug stay in the eye for a shorter time. Also, the static and dynamic barriers of the eyeball will reduce the bioavailability of drugs in the eye, thereby affecting the therapeutic effect. In addition, frequent dropping of corticosteroids may produce side effects such as corneal toxicity and elevated intraocular pressure. Therefore, it is one of the urgent problems to seek for a novel ophthalmic preparation that is safe and effective, can reduce side effects and administration frequency, and improve patient compliance. In another aspect, for some refractory corneal ulcers, conventional methods such as anti-inflammation, anti-infection, promotion of corneal repair, and amniotic membrane transplantation are not effective. Relevant innovative therapies for corneal healing have become the focus of increasing concern.

In order to solve the above problems, the present disclosure provides a pharmaceutical composition for corneal tissue repair by an insulin nanosystem and use thereof on the basis of the prior art.

### SUMMARY

An objective of the present disclosure is to provide a pharmaceutical composition for corneal tissue repair by an insulin nanosystem and use thereof. The present disclosure prepares a novel ophthalmic pharmaceutical composition by encapsulating insulin in nanomaterials like liposome. The ophthalmic pharmaceutical composition has the advantages of safety, effectiveness, low side effects and low administration frequency, and can effectively improve patient compliance; meanwhile, the ophthalmic pharmaceutical composition has important biological functions in anti-inflammation, anti-apoptosis and promoting nerve repair, and has more advantages in the treatment effects of anti-inflammation, anti-infection, and promoting corneal repair and strengthening amniotic membrane transplantation compared with a traditional treatment method.

The foregoing technical objective of the present disclosure is achieved through the following technical solutions:

A pharmaceutical composition for corneal tissue repair by an insulin nanosystem is prepared, including a nanomaterial-based drug delivery system and insulin encapsulated in the nanomaterial-based drug delivery system.

By adopting the above technical solution and utilizing unique properties of the nanomaterial, nanomaterials have more advantages than conventional drug delivery methods in delivering drugs for the treatment of eye diseases. Due to the advantages of excellent corneal penetrability, strong biocompatibility, non-toxicity, sustained release and long half-life mainly manifested in nanopreparations of drugs, a nano-controlled release system has an excellent application prospect in ophthalmology. In the present solution, after the insulin is encapsulated in liposome, an inner water phase of the liposome can protect the structure and conformation of the insulin, while an outer lipophilic layer helps improve absorption across the biomembrane barrier, and utilizes the biomembrane properties and drug delivery capabilities of the liposome to help improve bioavailability; and a liposome nano-controlled release system can effectively improve the ophthalmic drug delivery efficiency. The insulin has important biological functions in anti-inflammation, anti-apoptosis and promoting nerve repair. Encapsulating the insulin in the liposome can obtain a novel ophthalmic preparation that is safe and effective, with fewer side effects, low administration frequency and high patient compliance.

Further, the nanomaterial-based drug delivery system is a liposome.

The pharmaceutical composition for corneal tissue repair by an insulin nanosystem provided by the present disclosure is used in corneal repair.

Clinically, insulin is mainly used to treat diabetes. However, it has been shown that the insulin is not only an important metabolic regulating hormone, but also an important antiinflammatory factor, an anti-apoptotic factor, and a nerve repair factor, playing important biological roles in anti-inflammation, anti-infection, and promoting nerve repair; it has been shown that the insulin is involved in the regulation of inflammation and apoptosis by binding to receptors to activate the phosphatidylinositol-3-kinase (PI3K) pathway and the mitogen-activated protein kinase (MAPK) pathway; the insulin has protective effects on retinal Mǔller cells, nerves, and rat cortical neurons; topical use of insulin drops in patients with refractory neurotrophic corneal ulcers has found that complete corneal regeneration and repair can be achieved within 7-25 days. Therefore, the insulin has great potential in the treatment of corneal injury, not only avoiding adverse effects caused by long-term use of corticosteroids, but also having the advantages in promoting corneal repair, protecting and repairing corneal nerves.

To sum up, the present disclosure has the following beneficial effects:
1. The present disclosure prepares a novel ophthalmic pharmaceutical composition by encapsulating insulin in a nanomaterials like liposome, which has the advantages of safety, effectiveness, low side effects and low administration frequency, and can effectively improve patient compliance; and
2. the ophthalmic pharmaceutical composition has important biological functions in anti-inflammation, anti-apoptosis and promoting corneal cell and nerve repairs, and has more advantages in the treatment effects of anti-inflammation, anti-infection, promoting corneal repair and strengthening amniotic membrane transplantation compared with a traditional treatment method.

### BRIEF DESCRIPTION OF THE DRAWINGS

FIG. 1 illustrates insulin liposomes observed under a transmission electron microscope (TEM);

FIG. 2 illustrates repair of corneal injury in a normal saline group;

FIG. 3 illustrates repair of corneal injury in an insulin group;

FIG. 4 illustrates an INS solution, PFOB@LIP and INS/PFOB@LIP suspensions in the example of the present disclosure;

FIG. 5 is a TEM image of INS/PFOB@LIP (50 µg/mL) in the example of the present disclosure;

FIG. 6 illustrates the particle size distribution of PFOB@LIP in the example of the present disclosure;

FIG. 7 illustrates the particle size distribution of INS/PFOB@LIP in the example of the present disclosure;

FIG. 8 illustrates zeta potentials of PFOB@LIP and INS/PFOB@LIP in the example of the present disclosure;

FIG. 9 illustrates the ultraviolet absorption spectrum and absorption peak distribution (50 µg/mL) of INS, PFOB@LIP and INS/PFOB@LIP in the example of the present disclosure.

### DETAILED DESCRIPTION OF THE EMBODIMENTS

The present invention is further described in detail below with reference to accompanying drawings and an example.

Example: A pharmaceutical composition for corneal tissue repair by an insulin nanosystems included liposomes and insulin encapsulated in the liposomes. The pharmaceutical composition for corneal tissue repair by an insulin nanosystem could be used in corneal repair, and its preparation steps were as follows:
S1, soyabean lecithin, 1,2-distearoyl-sn-glycero-3-phosphoethanolamine-N-[methoxy(polyethylene glycol)-2000] (DSPE-PEG2000), and cholesterol were uniformly dissolved in chloroform to obtain a solution 1, and the solution 1 was placed into a flask, and a uniform liposome film was formed by rotary evaporation in a water bath.
S2, 2 mL of phosphate buffered saline (PBS) was added to the flask in step S1, and the liposome film was fully hydrated and eluted to obtain a film eluent.
S3, 5 mg of insulin was weighed, dissolved in 100 µL of dilute hydrochloric acid with a pH of 2, and 1 mL of PBS and 100 µL of perfluorooctyl bromide (PFOB) were added thereto to obtain a solution 2; the solution 2 was emulsified ultrasonically for 1 min to form a W/O type emulsion.
S4, the emulsion in step S3 was added to the film eluent for ultrasonic emulsification for 3 min to obtain nanoparticle liposomes; the nanoparticle liposomes were centrifuged at 6,000 rpm for 5 min, the supernatant was discarded, and the precipitates were collected and cryopreserved at 4° C. in the dark for later use.

As a highly efficient nano-controlled release system, insulin liposomes facilitate the realization of safe, effective and precise drug release. At the same time, the use of insulin can not only avoid the adverse reactions of long-term use of corticosteroids, but also can promote corneal repair and protect and repair corneal nerves; as a novel ophthalmic preparation, insulin liposomes can effectively reduce corneal toxicity, elevated intraocular pressure, and other side effects, and can effectively lower the administration frequency.

At present, tetrandrine-PFOB-loaded liposomes have been successfully prepared, and their characterization detection, along with analyses of safety concentration, and in vivo and in vitro accumulation and distribution, has been carried out. It has been found in the treatment of dry eyes in living rabbits that tetrandrine-PFOB-loaded liposomes have an obvious therapeutic effect on dry eyes and a lower effect on intraocular pressure.

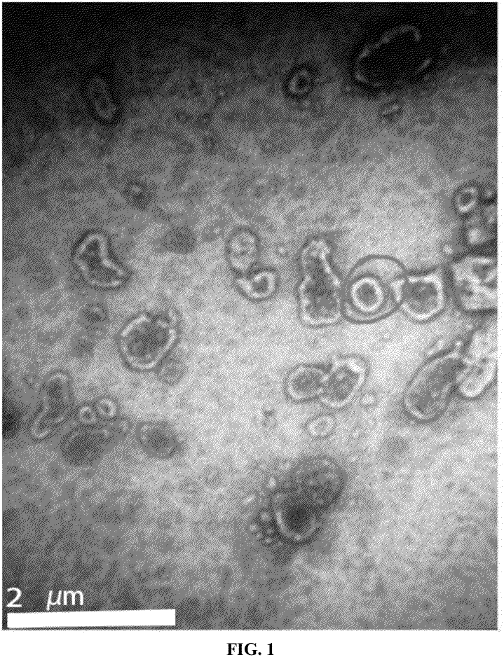

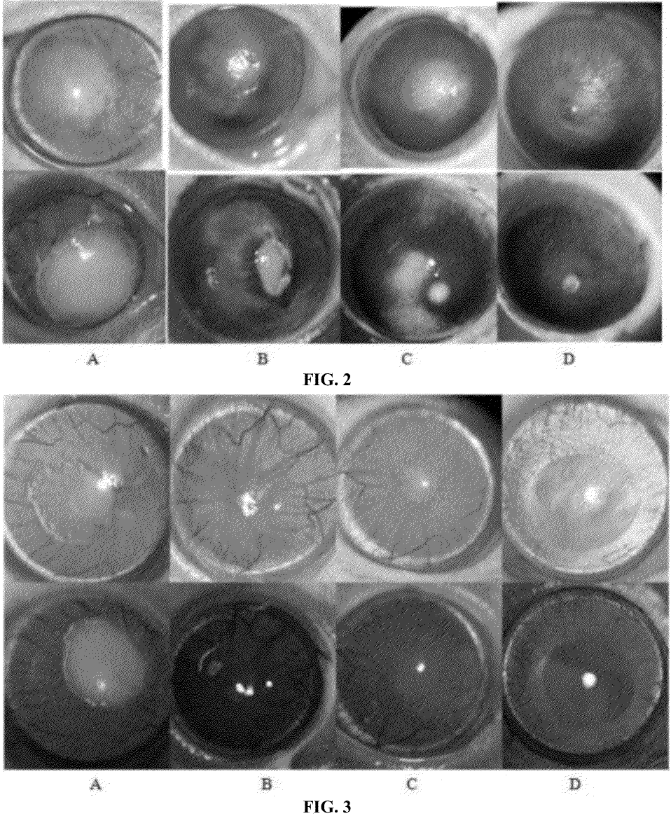

As shown in FIG. 1, insulin liposomes with a particle size of 500⁺ nm and a zeta potential of -50⁺ were prepared and used to treat alkali-burned rat corneas. As shown in FIGS. 2 to 3, alkali-burned rat corneas were treated with normal saline and insulin liposomes, respectively. In view of the repair of corneal injury in the normal saline group, panel A is an immediate photo of alkali burn; panel B shows hyphema on day 3; panel C shows neovascularization on day 7; and panel D shows substantial corneal epithelial healing with cornea perforation and massive neovascularization on day 14. In view of the repair of corneal injury in the insulin group, panel A is an immediate photo of alkali burn; panel B shows substantial corneal epithelial healing on day 3, with mild corneal edema; panel C shows complete corneal epithelial healing with vanished corneal edema on day 7; panel D shows complete corneal epithelial healing, no obvious corneal edema, and visible tiny neovascularization on day 14. Thus, it could be seen that the insulin group showed faster epithelial healing, fewer inflammatory responses, and less neovascularization than the normal saline group.

The present disclosure further detected the physicochemical properties of the insulin liposomes, and the steps were as follows:
A. Different concentrations of insulin-methanol solutions were prepared.
B. The absorbance was measured by an ultraviolet (UV) spectrophotometer, absorption peaks were determined according to the maximum absorbance value, and a concentration-absorbance standard curve was calculated.
C. The absorbance curves of INS, PFOB@LIP, and INS/PFOB@LIP were detected by the UV spectrophotometer, and their respective characteristic peaks were observed, where INS represents insulin and LIP represents lipid.
D. The morphology of the INS/PFOB@LIP was observed under TEM.
E. The particle sizes and zeta potentials of the PFOB@LIP and INS/PFOB@LIP were measured by Malvern Zetasizer.

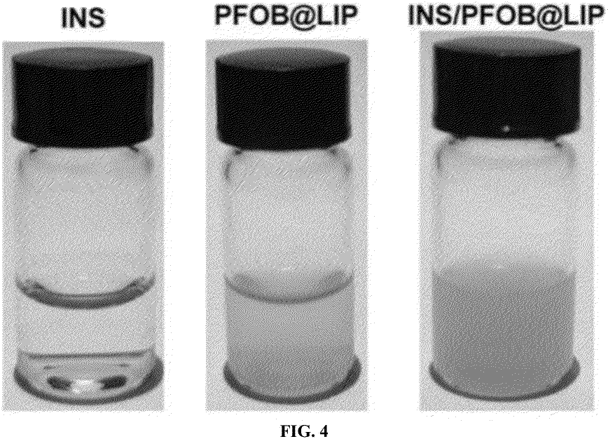

As shown in FIG. 4, PFOB@LIP and INS/PFOB@LIP were dissolved in PBS to prepare 2 mL of 400 µg/mL solutions, respectively. The INS solution (200 µg/mL, 2 mL) was colorless and transparent. PFOB@LIP and INS/PFOB@LIP suspensions were milky white, and the INS/PFOB@LIP suspension was darker.

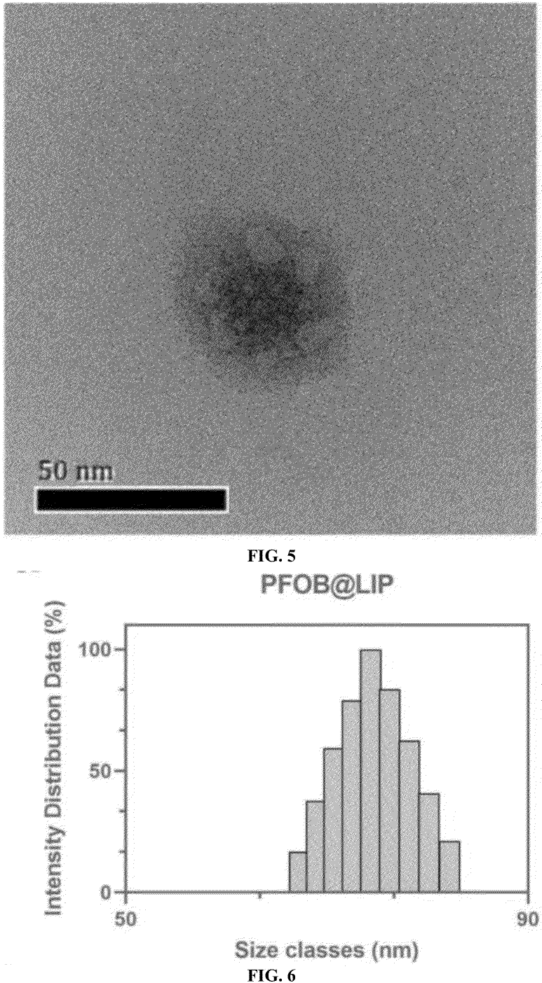

As shown in FIG. 5, TEM showed that the INS/PFOB@LIP had a relatively uniform and consistent spherical appearance, the liposome was a shell-core structure, and a phospholipid bilayer could be observed.

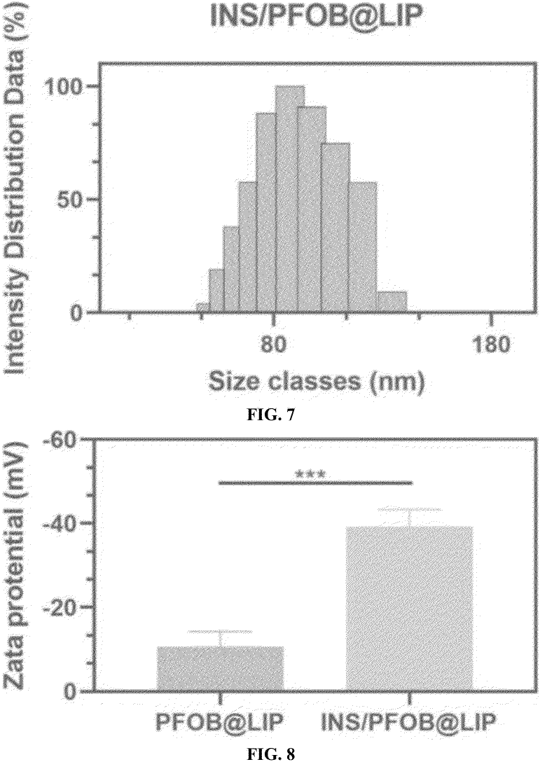

As shown in FIGS. 6 and 7, the average particle size of the PFOB@LIP was (74.68 ± 6.51) nm, and that of the INS/PFOB@LIP was (87.41 ± 4.62) nm.

As shown in FIG. 8, the zeta potentials of the PFOB@LIP and INS/PFOB@LIP were (-10.70 ± 2.90) mV and (-39.27 ± 3.32) mV, respectively. The difference in zeta potential was statistically significant between the PFOB@LIP and the INS/PFOB@LIP (P < 0.001).

The present disclosure further detected the encapsulation efficiency and drug loading capacity of the insulin liposome, and test methods were as follows: a quantity of insulin liposomes were prepared, and the drug encapsulation efficiency and drug loading capacity were determined by the demulsification method; insulin encapsulation efficiency = drug loading capacity / total input × 100%; drug loading capacity of insulin = drug loading capacity / total amount of liposomes × 100%.

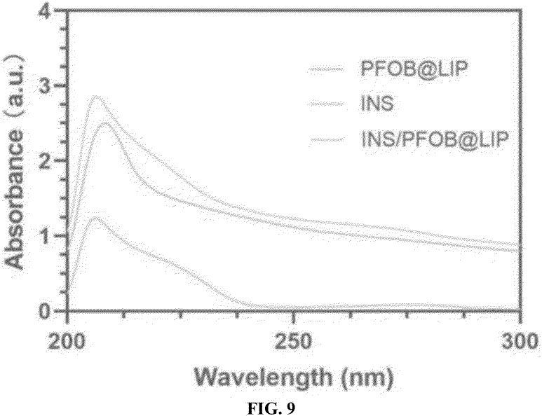

As shown in FIG. 9, the UV spectrophotometer showed that the INS and the INS/PFOB@LIP had characteristic absorption peaks at 205 nm, while the PFOB@LIP did not have such a characteristic, demonstrating that the INS/PFOB@LIP successfully encapsulated the INS; through the standard curve of the ultraviolet absorption spectrum of the INS, it could be expressed by the following linear regression equation: Y = 0.01658 × X - 0.1114, R² = 0.9950. The encapsulation efficiency and drug loading capacity of the insulin (INS) were calculated as (32.86 ± 1.24)% and (16.43 ± 1.24)%, respectively.

In the foregoing example of the present disclosure, a novel ophthalmic pharmaceutical composition is prepared by encapsulating insulin in nanomaterials like liposome. The ophthalmic pharmaceutical composition has the advantages of safety, effectiveness, low side effects and low administration frequency, and can effectively improve patient compliance; meanwhile, the ophthalmic pharmaceutical composition has important biological functions in anti-inflammation, anti-apoptosis and promoting nerve repair, and has more advantages in the treatment effects of anti-inflammation, anti-infection, and promoting corneal repair and strengthening amniotic membrane transplantation compared with a traditional treatment method.

This specific example is only an explanation of the present disclosure, but it is not a limitation of the present disclosure. After reading this specification, those skilled in the art can make modifications without creative contribution to the present example as needed, but they are protected by patent law as long as these modifications fall within the scope of the present disclosure.

1. A pharmaceutical composition for corneal tissue repair by an insulin nanosystem, comprising a nanomaterial-based drug delivery system and insulin encapsulated in the nanomaterial-based drug delivery system.

2. The pharmaceutical composition for corneal tissue repair by an insulin nanosystem according to claim 1, wherein the nanomaterial-based drug delivery system is a liposome.

3. A method for corneal tissue repair , comprising administering the pharmaceutical composition for corneal tissue repair by an insulin nanosystem according to claim 1 to a subject in demand .
